(19) United States
(12) Patent Application Publication
(10) Pub. No.: US 2007/0184001 A1
(43) Pub. Date: Aug. 9, 2007

(54) Cosmetic composition comprising a nonionic fixing polymer and a specific ester of polyethylene glycol and of fatty acid, and a method for fixing the hairstyle

(76) Inventors: Sabine Vrignaud, Ia Plaine (FR); Cecile Bebot, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/655,862

(22) Filed: Jan. 22, 2007

Related U.S. Application Data
(60) Provisional application No. 60/771,040, filed on Feb. 8, 2006.

(30) Foreign Application Priority Data
Jan. 20, 2006 (FR) 06 00557

(51) Int. Cl. A61K 8/31

(52) U.S. Cl. 424/70.2; 424/70.11; 424/70.14; 424/70.9

(57) ABSTRACT

The present disclosure relates to a cosmetic composition comprising, in a cosmetically acceptable medium, at least one nonionic fixing polymer other than poly(vinylpyrrolidone/vinyl acetate) and at least one ester of polyethylene glycol and of fatty acid of formula:
R₁CO—(OCH₂CH₂)ₙ₀—[OCH₂—CH(OR₂)—CH₂]ₙ₁—(OCH₂CH₂)ₙ₂—R₃ wherein
R₂ is chosen from a hydrogen atom and a (CH₂CH₂O)ₙ₃COR₄ group; n1 is an integer equal to 0 or 1; n2 is an integer ranging from 2 to 300; n3 is an integer ranging from 1 to 300; n0 is an integer ranging from 0 to 300;
R₃ is chosen from a hydrogen atom, an OH group and a R₅COO group;
R₁, R₄ and R₅ are chosen, independently of one another, from C₁₀ to C₃₀ alkyl groups and C₁₀ to C₃₀ alkylene groups. The present disclosure is also targeted at a method for the shaping or the form retention of the hairstyle comprising applying the cosmetic composition to hair, for example, for the fixing and the shaping of the hairstyle in a lasting manner.

This application claims benefit of U.S. Provisional Application No. 60/771,040, filed Feb. 8, 2006, the contents of which are incorporated herein by reference. This application also claims benefit of priority under 35 U.S.C. § 119 to French Patent Application No. FR 06 00557, filed Jan. 20, 2006, the contents of which are also incorporated herein by reference.

The present disclosure relates to a styling cosmetic composition and to a method for fixing keratinous fibers, e.g., human keratinous fibers, such as the hair.

The most widespread cosmetic compositions on the cosmetic market for hair shaping and/or form retention of the hairstyle are typically compositions essentially composed of a solution, generally an alcoholic or aqueous/alcoholic solution, and of at least one component referred to as a fixing component. These fixing components are generally polymeric resins, the role of which is to form joins between the hairs, and are often formulated as a mixture with various cosmetic adjuvants.

The compositions can also be provided in the form of gels.

Cosmetic compositions may be packaged either in a pot or in a tube, or in a pump-action spray, or in an appropriate aerosol container pressurized using a propellant. The aerosol system then comprises, on the one hand, a liquid phase (or dispensable) and, on the other hand, a propellant.

Once applied to the hair, the composition comprising the fixing components and an appropriate solvent dries, making possible the formation of joins necessary for the fixing of the hair by the fixing components. The joins have to be sufficiently rigid to provide form retention of the hair; however, they must also be sufficiently weak for the user to be able, by combing or by brushing the hair, to destroy them without hurting the scalp or damaging the hair.

The conventional film-forming resins generally used as fixing agents in an alcoholic medium may exhibit the disadvantage of conferring mediocre cosmetic properties on a styling composition; for instance, the feel obtained by the use of the compositions based on film-forming resins is not always satisfactory.

Generally, fixing polymers make it possible to fix a hairstyle in the desired shape. However, during the course of a day, a hairstyle is subjected to various deformations (passing of the hand through the hair, wearing of a hood, and the like) which, little by little, cause the polymer film to crumble and reduce the hold of the hairstyle.

The present inventors have now discovered that the combination of at least one nonionic fixing polymer and of at least one specific ester of polyethylene glycol and of fatty acid makes it possible to satisfactorily fix the hairstyle and also to confer on it a shape which lasts longer than the hairstyles shaped using a conventional fixing composition.

These compositions, as disclosed herein, also make it possible to confer satisfactory cosmetic properties on the hair.

Other characteristics, aspects, objects and benefits of the present disclosure will become even more clearly apparent upon reading the description and examples which follow.

The present disclosure relates to a cosmetic composition comprising, in a cosmetically acceptable medium, at least one nonionic fixing polymer other than poly(vinylpyrrolidone/vinyl acetate) copolymers and at least one specific ester of polyethylene glycol and of fatty acid.

In one embodiment of the present disclosure, the cosmetic composition is a hair composition, such as a styling hair cosmetic composition.

According to the present disclosure, the cosmetic composition may be provided in the form of a spray, of a foam or of a gel.

Additionally, the present disclosure also comprises a process for the shaping or form retention of the hairstyle wherein the cosmetic composition, as disclosed herein, is employed.

The present disclosure also relates to a method for fixing and retention of the hair using the presently disclosed cosmetic composition as a styling composition for the fixing and form retention of the hair, such as for conferring good hold on the hairstyle over time.

In at least one embodiment of the present disclosure, the compositions may be non-washing (non-detergent) compositions, comprising, for example, less than 4% by weight of detergent surfactants and further, for example, less than 1% by weight of detergent surfactants, with respect to the total weight of the composition. In another embodiment, the presently disclosed composition does not comprise any detergent surfactants.

As used herein, the term “detergent surfactants” is understood to mean any anionic or nonionic surface-active agent, different from the esters of polyethylene glycol and of fatty acid disclosed herein.

The cosmetically acceptable medium used in the presently disclosed compositions is an aqueous/alcoholic or alcoholic medium. The alcohol used may be chosen from a monohydroxylated alcohol and/or a polyol. For instance, the monohydroxylated alcohol may be chosen from lower C₁-C₄ alcohols, such as ethanol, isopropanol, tert-butanol or n-butanol. In at least one embodiment, the alcohol used is ethanol.

Non-limiting examples of the polyols which may be used in the presently disclosed compositions include propylene glycol, polyethylene glycols, polyol ethers and their mixtures. Alcohol may be present in the compositions of the present disclosure in an amount ranging from 0.1 to 99%, for instance, from 0.5 to 50%, and further, for instance, from 1 to 30%, by weight, with respect to the total weight of the composition.

In one embodiment of the present disclosure, alcohol is present in an amount ranging from 1 to 25% by weight, with respect to the total weight of the composition.

The esters of polyethylene glycol and of fatty acid which may be used in the compositions disclosed herein correspond to the formula:
R₁CO—(OCH₂CH₂)ₙ₀—[OCH₂—CH(OR₂)—CH₂]ₙ₁—(OCH₂CH₂)ₙ₂—R₃
wherein

R₂ is chosen from hydrogen and a (CH₂CH₂O)ₙ₃COR₄ group;

n₁ is an integer equal to 0 or 1;

n₂ is an integer ranging from 2 to 300;

n₃ is an integer ranging from 1 to 300;

n₀ is an integer ranging from 0 to 300;

R₃ is chosen from hydrogen, an OH group and an R₅COO group;

R₁, R4 and R₅ are chosen from, independently of one another, C₁₀ to C₃₀ alkyl groups and C₁₀ to C₃₀ alkylene groups.

Non-limiting examples of esters of polyethylene glycol and of fatty acid useful herein include polyethylene glycol (150 EO) distearate and glyceryl oxyethylene (200 EO) monostearate.

The at least one ester of polyethylene glycol and of fatty acid is present in the composition in an amount ranging from 0.01% to 20% by weight, for example, from 0.1% to 15% by weight and further for example, from 1% to 10% by weight, with respect to the total weight of the composition.

Any nonionic fixing polymer and mixtures thereof used in the hair field may be used in the compositions disclosed herein.

In at least one embodiment, the nonionic fixing polymers useful herein may be chosen, for example, from:
polyalkyloxazolines;
vinyl acetate homopolymers;
vinyl acetate copolymers other than poly(vinylpyrrolidone/vinyl acetate) copolymers, such as, for example, copolymers of vinyl acetate and of acrylic ester, copolymers of vinyl acetate and of ethylene, or copolymers of vinyl acetate and of maleic ester, for example of dibutyl maleate;
acrylic ester homopolymers and copolymers, such as, for example, copolymers of alkyl acrylates and of alkyl methacrylates, such as the products provided by Röhm & Haas under the names Primal® AC-261 K and Eudragit® NE 30 D, by BASF under the name 8845 or by Hoechst under the name Appretan® N9212;
copolymers of acrylonitrile and of a nonionic monomer chosen, for example, from butadiene and alkyl (meth)acrylates; mention may be made of the products provided under the name CJ 0601 B by Röhm & Haas;
styrene homopolymers;
styrene copolymers, such as, for example, copolymers of styrene and of alkyl (meth)acrylate, such as the products Mowilith® LDM 6911, Mowilith® DM 611 and Mowilith® LDM 6070 provided by Hoechst or the products Rhodopas® SD 215 and Rhodopas® DS 910 provided by Rhone-Poulenc, copolymers of styrene, of alkyl methacrylate and of alkyl acrylate, copolymers of styrene and of butadiene or copolymers of styrene, of butadiene and of vinylpyridine;
polyamides;
vinyllactam homopolymers, such as vinylpyrrolidone homopolymers and such as the polyvinylcaprolactam sold under the name Luviskol® Plus by BASF; and
vinyllactam copolymers, such as the poly(vinylpyrrolidone/vinyllactam) copolymer sold under the trade name Luvitec® VPC 55K65W by BASF.

The alkyl groups of the nonionic polymers mentioned above comprise, in at least one embodiment, 1 to 6 carbon atoms.

According to the present disclosure, it is also possible to use fixing polymers of nonionic grafted silicone type comprising a polysiloxane part and a part composed of a non-silicone organic chain, one of the two parts constituting the main chain of the polymer and the other being grafted onto the main chain.

These polymers are disclosed, for example, in European Patent Application Nos.-0 412 704, 0 412 707, 0 640 105, 0 582 152, International Patent Application Nos. 95/00578 and 93/23009 and U.S. Pat. Nos. 4,693,935, 4,728,571 and 4,972,037.

It is also possible to use, as fixing polymers, nonionic, silicone or non-silicone, functionalized or nonfunctionalized polyurethanes or their mixtures.

Generally, the at least one nonionic fixing polymer is present in an amount ranging from 0.1 to 20%, for example from 1 to 15% by weight, and further for example, from 7 to 15% by weight, of the total weight of the composition. In one embodiment, this content ranges from 7 to 15% by weight of the total weight of the composition

In at least one embodiment of the present disclosure, the ratio of the at least one nonionic fixing polymer to the at least one ester of polyethylene glycol and of fatty acid by weight of the compositions disclosed herein is greater than 0.6, for example, from 0.6 to 10.

The compositions according to the present disclosure may also comprise at least one additional anionic, cationic, amphoteric or nonionic fixing polymer other than the specific nonionic fixing polymers of poly(vinylpyrrolidone/vinyl acetate) type described above.

The at least one additional fixing polymer may be present in an amount ranging from 0.1 to 20%, such as from 1 to 15%, by weight, of the total weight of the composition.

According to at least one embodiment of the present disclosure, the composition also comprises at least one ionic or nonionic thickening agent, also referred to as a “rheology-adjusting agent”.

The rheology-adjusting agents useful herein may be chosen from fatty acid amides (coconut diethanol- or monoethanolamide, oxyethylenated alkyl ether carboxylic acid monoethanolamide). Non-limiting mention may be made of the following polymeric rheology-adjusting agents: cellulose thickeners (hydroxyethylcellulose, hydroxypropylcellulose, carboxymethylcellulose), guar gum and its derivatives (hydroxypropyl guar), gums of microbial origin (xanthan gum, scleroglucan gum), crosslinked homopolymers or copolymers of acrylic acid or of acrylamidopropanesulphonic acid, and associative thickening polymers, such as described below. In one embodiment, the polymeric agent can be chosen from crosslinked homopolymers or copolymers of acrylic acid or of acrylamidopropanesulphonic acid, and for instance from crosslinked homopolymers or copolymers of acrylamidopropanesulphonic acid.

The at least one thickening agent may be present in the compositions disclosed herein in an amount ranging from 0.01 to 10%, for instance from 0.1 to 5% and further for instance from 0.3 to 3%, by weight, with respect to the total weight of the composition.

As used herein, the term “associative polymers” is understood to mean water-soluble polymers capable, in an aqueous medium, of reversibly associating with one another or with other molecules. The chemical structure of associative polymers comprises hydrophilic regions and hydrophobic regions characterized by at least one fatty chain.

When the thickening agent used herein is an associative polymer, such polymers may be chosen from anionic, cationic, amphoteric and nonionic associative polymers, and may be present in the compositions in an amount ranging from 0.01 to 10%, such as from 0.1 to 5%, by weight, with respect to the total weight of the composition.

Anionic Associative Polymers

Non-limiting mention may be made, among anionic associative polymers, of:
(I) those comprising at least one hydrophilic unit and at least one allyl ether unit comprising a fatty chain, for instance, those for which the hydrophilic unit is composed of an ethylenic unsaturated anionic monomer, further for instance of a vinyl carboxylic acid and even further, for instance, of an acrylic acid or a methacrylic acid or the mixtures of these, and for which the allyl ether unit comprising a fatty chain corresponds to the monomer of following formula (XV):
   CH₂=CR′CH₂OBₙR (XV)
   wherein R′ is chosen from an H atom and a CH₃ group, B is the ethyleneoxy radical, n is zero or is an integer ranging from 1 to 100, and R is a hydrocarbon radical chosen from alkyl, arylalkyl, aryl, alkylaryl,and cycloalkyl radicals comprising from 8 to 30 carbon atoms, for instance from 10 to 24, and further for instance from 12 to 18 carbon atoms. In at least one embodiment, formula (XV) is a unit wherein R′ is H, n is equal to 10 and R is a stearyl (C₁₈) radical.

Non-limiting examples of anionic associative polymers of this type are disclosed and prepared, according to an emulsion polymerization process, in European Patent No. 0 216 479.

According to at least one embodiment, the anionic associative polymers used are those formed from 20 to 60% by weight of acrylic acid and/or methacrylic acid, from 5 to 60% by weight of lower alkyl (meth)acrylates, from 2 to 50% by weight of allyl ether comprising a fatty chain of formula (XV), and from 0 to 1% by weight of a crosslinking agent which is a known copolymerizable polyethylenic unsaturated monomer, such as diallyl phthalate, allyl (meth)acrylate, divinylbenzene, (poly)ethylene glycol dimethacrylate and methylenebisacrylamide.

In a further embodiment, the anionic associative polymers may be chosen from crosslinked terpolymers of methacrylic acid, of ethyl acrylate and of polyethylene glycol (10 EO) ether of stearyl alcohol (Steareth 10), such as those sold by Allied Colloids under the names Salcare SC 80® and Salcare SC90®, which are 30% aqueous emulsions of a crosslinked terpolymer of methacrylic acid, of ethyl acrylate and of steareth-10 allyl ether (40/50/10).
(II) those comprising at least one hydrophilic unit of olefinic unsaturated carboxylic acid type and at least one hydrophobic unit of alkyl (C₁₀-C₃₀) ester of unsaturated carboxylic acid type.

In at least one embodiment, such polymers may be chosen from those for which the hydrophilic unit of olefinic unsaturated carboxylic acid type corresponds to the monomer of following formula (XVI): 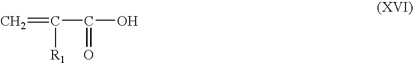
wherein R₁ is chosen from H, a CH₃ group and a C₂H₅ group (i.e., acrylic acid, methacrylic acid or ethacrylic acid units), and for which the hydrophobic unit of alkyl (C₁₀-C₃₀) ester of unsaturated carboxylic acid type corresponds to the monomer of following formula (XVII): 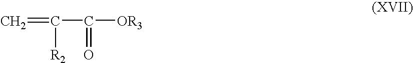
wherein R₂ is chosen from H, a CH₃ group and a C₂H₅ group (i.e., acrylate, methacrylate or ethacrylate units) and, in one embodiment, from H (acrylate units) and CH₃ (methacrylate units), and wherein R₃ is a C₁₀-C₃₀, such as a C₁₂-C₂₂, alkyl radical.

Alkyl (C₁₀-C₃₀) esters of unsaturated carboxylic acids in accordance with the present disclosure, comprise, for example, lauryl acrylate, stearyl acrylate, decyl acrylate, isodecyl acrylate, dodecyl acrylate, and the corresponding methacrylates, lauryl methacrylate, stearyl methacrylate, decyl methacrylate, isodecyl methacrylate and dodecyl methacrylate.

Anionic polymers of this type are, for example, disclosed and prepared according to U.S. Pat. Nos. 3,915,921 and 4,509,949.

In at least one embodiment, the anionic associative polymers may be formed from a mixture of monomers comprising:
(i) essentially acrylic acid,
(ii) an ester of formula (XVII) described above and wherein R₂ is a hydrogen atom or CH₃ group, R₃ is an alkyl radical comprising 12 to 22 carbon atoms, and
(iii) a crosslinking agent which is a known copolymerizable polyethylenic unsaturated monomer, such as diallyl phthalate, allyl (meth)acrylate, divinylbenzene, (poly)ethylene glycol dimethacrylate and methylenebisacrylamide.

In at least one embodiment, anionic associative polymers of this type may be chosen from those composed of 95 to 60% by weight of acrylic acid (hydrophilic unit), 4 to 40% by weight of C₁₀-C₃₀ alkyl acrylate (hydrophobic unit) and 0 to 6% by weight of crosslinking polymerizable monomer or else those composed of 98 to 96% by weight of acrylic acid (hydrophilic unit), 1 to 4% by weight of C₁₀-C₃₀ alkyl acrylate (hydrophobic unit) and 0.1 to 0.6% by weight of crosslinking polymerizable monomer such as those described above.

In one embodiment, the anionic associative polymers formed from a mixture of monomers are chosen from the products sold by Goodrich under the tradenames Pemulen TR1®, Pemulen TR2® or Carbopol 1382® and the product sold by S.E.P.P.I.C. under the name Coatex SX®. In at least one further embodiment, Pemulen TR1® is used.
(III) maleic anhydride/C₃₀-C₃₈ α-olefin/alkyl maleate terpolymers, such as the product (maleic anhydride/C₃₀-C₃₈ α-olefin/isopropyl maleate copolymer) sold under the name Performa V 1608® by Newphase Technologies.
(IV) acrylic terpolymers comprising:

(a) 20% to 70% by weight of a carboxylic acid comprising α,β-monoethylenic unsaturation,

(b) 20 to 80% by weight of a non-surface-active monomer comprising α,β-monoethylenic unsaturation other than (a),

(c) 0.5 to 60% by weight of a nonionic monourethane which is the reaction product of a monohydric surfactant with a monoisocyanate comprising monoethylenic unsaturation,

such as those disclosed in European Patent Application No. 0 173 109 and for example that disclosed in Example 3, namely a methacrylic acid/methyl acrylate/dimethyl(meta-isopropenyl)benzyl isocyanate terpolymer of ethoxylated behenyl alcohol (40 EO) as a 25% aqueous dispersion.
(V) copolymers comprising, among their monomers, a carboxylic acid comprising α,β-monoethylenic unsaturation and an ester of a carboxylic acid comprising α,β-monoethylenic unsaturation and of an oxyalkylenated fatty alcohol. In at least one embodiment these compounds also comprise, as monomer, an ester of a carboxylic acid comprising α,β-monoethylenic unsaturation and of a C₁-C₄ alcohol. Non-limiting mention may be made, as example of this type of compound, of Aculyn 22®, sold by Röhm & Haas, which is a methacrylic acid/ethyl acrylate/oxyalkylenated stearyl methacrylate terpolymer.
   Cationic Associative Polymers

Non-limiting mention maybe made, among cationic associative polymers useful herein, of:
(I) associative cationic polyurethanes, the family of which is disclosed in French Patent Application No. 00/09609, now published as FR 2 811 993, and which is represented by the following formula (XVIII):
   R—X—(P)ₙ—[L-(Y)ₘ]ᵣ-L′-(P′)ₚ—X′—R′ (XVIII)
   wherein:

R and R′, which are identical or different, are chosen from a hydrophobic group and a hydrogen atom;

X and X′, which are identical or different, are groups comprising an amine functional group which may or may not carry a hydrophobic group, or alternatively the L″ group;

L, L′ and L″, which are identical or different, are groups derived from diisocyanates;

P and P′, which are identical or different, are groups comprising an amine functional group which may or may not carry a hydrophobic group;

Y is a hydrophilic group;

r is an integer ranging from 1 to 100, for example from 1 to 50 and further for example from 1 to 25,

n, m, and p have values, each independently of the others, ranging from 0 to 1 000;

the molecule comprising at least one protonated or quaternized amine functional group and at least one hydrophobic group.

In at least one embodiment of the cationic associative polyurethanes, the sole hydrophobic groups are the R and R′ groups at the chain ends.

According to the present disclosure, an example of a family of cationic associative polyurethanes is that corresponding to the formula (XVIII) above wherein:
R and R′ are both independently a hydrophobic group,
X and X′ are each an L″ group,
n and p have values ranging from 1 to 1000, and
L, L′, L″, P, P′, Y and m have the meanings indicated above.

Another example of a family of cationic associative polyurethanes is that corresponding to the formula (XVIII) above wherein R and R′ are both independently a hydrophobic group, X and X′ are each an L″ group, n and p have the value 0 and L, L′, L″, Y and m have the meanings indicated above. The fact than n and p have the value 0 means that these polymers do not comprise units derived from a monomer comprising an amine functional group incorporated in a polymer during the polycondensation. The protonated amine functional groups of these polyurethanes result from the hydrolysis of isocyanate functional groups, in excess, at the chain end, followed by the alkylation of the primary amine functional groups formed by alkylating agents comprising a hydrophobic group, that is to say compounds of RQ or R′Q type wherein R and R′ are as defined above and Q is a leaving group, such as a halide, a sulphate, and the like.

Yet another example of a family of cationic associative polyurethanes is that corresponding to the formula (XVIII) above wherein:
R and R′ are both independently a hydrophobic group,
X and X′ are both independently a group comprising a quaternary amine, n and p have the value zero, and L, L′, Y and m have the meanings indicated above.

The number-average molecular weight of the cationic associative polyurethanes may range from 400 to 500 000, and further for example, from 1000 to 400 000 and even further from 1000 to 300 000.

As used herein, the term “hydrophobic group” is understood to mean a radical or polymer comprising a saturated or unsaturated and linear or branched hydrocarbon chain, which can comprise at least one heteroatom, such as P, O, N, or S, or a radical comprising a perfluorinated or silicone chain. When it denotes a hydrocarbon radical, the hydrophobic group comprises at least 10 carbon atoms, for instance from 10 to 30 carbon atoms, further for instance from 12 to 30 carbon atoms and even further for instance from 18 to 30 carbon atoms.

In one embodiment of the present disclosure, the hydrocarbon group originates from one monofunctional compound.

By way of example, the hydrophobic group may result from a fatty alcohol, such as stearyl alcohol, dodecyl alcohol or decyl alcohol. It may also be chosen from a hydrocarbon polymer, such as, for example, polybutadiene.

When X and/or X′ are chosen from a group comprising a tertiary or quaternary amine, X and/or X′ can represent one of the following formulae: 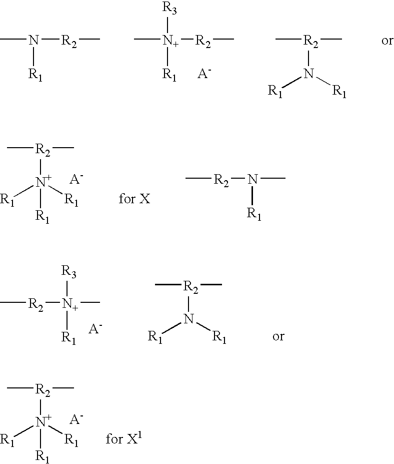
wherein:

R₂ is chosen from a linear or branched alkylene radical comprising from 1 to 20 carbon atoms, which may or may not comprise a saturated or unsaturated ring, and from an arylene radical, it being possible for at least one of the carbon atoms to be replaced by a heteroatom chosen from N, S, O and P;

R₁ and R₃, which are identical or different, are chosen from linear or branched C₁-C₃₀ alkyl radicals, linear or branched C₁-C₃₀ alkenyl radicals and aryl radicals, it being possible for at least one of the carbon atoms to be replaced by a heteroatom chosen from N, S, O or P;

A⁻ is a physiologically acceptable counterion.

The L, L′ and L″ groups represent a group of formula: 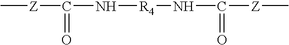
wherein:
Z is —O—, —S— or —NH—; and

R₄ is chosen from a linear or branched alkylene radical comprising 1 to 20 carbon atoms, which may or may not comprise a saturated or unsaturated ring, and from an arylene radical, it being possible for at least one of the carbon atoms to be replaced by a heteroatom chosen from N, S, O and P.

The P and P′ groups, comprising an amine functional group, may represent at least one of the following formulae: 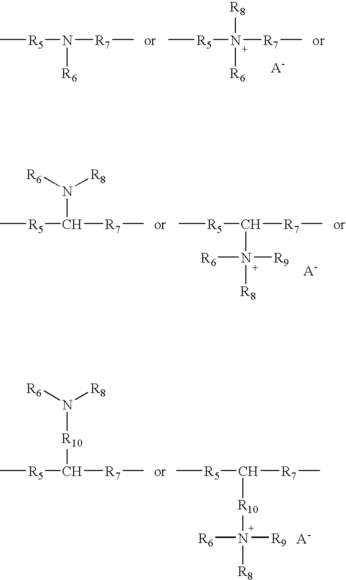
wherein:

R₅ and R₇ have the same meanings as R₂ defined above;

R₆, R₈ and R₉ have the same meanings as R₁ and R₃ defined above;

R₁₀ is chosen from a linear or branched alkylene group which is optionally unsaturated and which can comprise at least one heteroatom chosen from N, O, S and P,
and A⁻ is a physiologically acceptable counterion.

As indicated above and used herein, Y is a “hydrophilic group,” wherein the term “hydrophilic group” is understood to mean a water-soluble polymeric or nonpolymeric group.

Non-limiting mention may be made, by way of example, when polymers are not concerned, of ethylene glycol, diethylene glycol and propylene glycol.

Non-limiting examples of the hydrophilic polymer may include polyethers, sulphonated polyesters, sulphonated polyamides or a blend of these polymers and non-limiting examples of the hydrophilic compound may include a polyether and for instance a poly(ethylene oxide) or poly(propylene oxide).

The cationic associative polyurethanes of formula (XVIII) which may be used according to the present disclosure, are formed from diisocyanates and from various compounds having functional groups comprising a labile hydrogen. The functional groups comprising a labile hydrogen may be alcohol, primary or secondary amine or thiol functional groups which give, after reaction with the diisocyanate functional groups, polyurethanes, polyureas and polythioureas respectively. As used herein, the term “polyurethanes” is understood to encompass three types of polymers, namely polyurethanes proper, polyureas and polythioureas, and copolymers of these.

A first type of compound participating in the preparation of the polyurethane of formula (XVIII) is a compound comprising at least one unit comprising an amine functional group. This compound can be polyfunctional but in at least one embodiment is difunctional, that is to say that this compound comprises two labile hydrogen atoms carried, for example, by the hydroxyl, primary amine, secondary amine or thiol functional group. Additionally, a mixture of polyfunctional and difunctional compounds may be used in the preparation of the polyurethane of formula (XVIII) wherein the percentage of polyfunctional compounds is low.

As indicated above, this first type of compound participating in the preparation of the polyurethane of formula (XVIII) may comprise at least one unit comprising an amine functional group. It is then a polymer carrying a repetition of the unit comprising an amine functional group.

The compounds of this type participating in the preparation of the polyurethane of formula (XVIII) may be chosen from one of the following formulae:
HZ—(P)ₙ—ZH,
or
HZ—(P′)ₚ—ZH
wherein Z, P, P′, n and p are as defined above.

Non-limiting mention may be made, as example of compound comprising an amine functional group, of N-methyldiethanolamine, N-tert-butyldiethanolamine or N-sulphoethyldiethanolamine.

The second compound participating in the preparation of the polyurethane of formula (XVIII) is a diisocyanate corresponding to the formula:
O═C═N—R₄—N═C═O
wherein R₄ is defined above.

Non-limiting mention may be made, by way of example, of methylenediphenyl diisocyanate, methylenecyclohexane diisocyanate, isophorone diisocyanate, toluene diisocyanate, naphthalene diisocyanate, butane diisocyanate or hexane diisocyanate.

A third compound which participates in the preparation of the polyurethane of formula (XVIII) is a hydrophobic compound intended to form the end hydrophobic groups of the polymer of formula (XVIII).

This compound is composed of a hydrophobic group and of a functional group comprising a labile hydrogen, for example a hydroxyl, primary or secondary amine or thiol functional group.

By way of example, this compound can be a fatty alcohol, such as stearyl alcohol, dodecyl alcohol or decyl alcohol. When this compound comprises a polymer chain, it may, for example, be α-hydroxyl hydrogenated polybutadiene.

The hydrophobic group of the polyurethane of formula (XVIII) may also result from the quaternization reaction of the tertiary amine of the compound comprising at least one tertiary amine unit. Thus, the hydrophobic group is introduced by the quaternizing agent. This quaternizing agent is a compound of RQ or R′Q type wherein R and R′ are as defined above and Q is a leaving group, such as a halide, a sulphate, and the like.

As disclosed herein, the cationic associative polyurethane may additionally comprise a hydrophilic sequence. This sequence is contributed by a fourth type of compound participating in the preparation of the polymer. This compound may, for example, be polyfunctional, and further for instance, difunctional. It is also possible to have a mixture where the percentage of polyfunctional compound is low.

The functional groups comprising a labile hydrogen are alcohol, primary or secondary amine or thiol functional groups. This compound can be a polymer terminated at the ends of the chains by one of these functional groups comprising a labile hydrogen.

Non-limiting mention may be made, by way of example, when polymers are not concerned, of ethylene glycol, diethylene glycol and propylene glycol.

Non-limiting mention may be made, by way of example, of the following hydrophilic polymers: polyethers, sulphonated polyesters, sulphonated polyamides or a blend of these polymers. For example, the hydrophilic compound may be a polyether and further, for example, a poly(ethylene oxide) or poly(propylene oxide).

According to at least one embodiment of the present disclosure, the hydrophilic group recorded as Y in the formula (XVIII) is optional. This is because the units comprising a quaternary or protonated amine functional group may be sufficient to contribute the solubility or water-dispersibility necessary for this type of polymer in an aqueous solution.

Although the presence of a hydrophilic Y group is optional, the cationic associative polyurethanes may comprise such a group.
(II) quaternized cellulose derivatives and polyacrylates comprising noncyclic aminated side groups.

The quaternized cellulose derivatives comprise,
quaternized celluloses modified by groups comprising at least one fatty chain, such as alkyl, arylalkyl or alkylaryl groups comprising at least 8 carbon atoms, or mixtures of these,
quaternized hydroxyethylcelluloses modified by groups comprising at least one fatty chain, such as alkyl, arylalkyl or alkylaryl groups comprising at least 8 carbon atoms, or mixtures of these.

The alkyl radicals carried by the above quaternized celluloses or hydroxyethylcelluloses comprise 8 to 30 carbon atoms. The aryl radicals may be chosen from the phenyl, benzyl, naphthyl or anthryl groups.

Non-limiting examples of quaternized alkylhydroxyethylcelluloses comprising C₈-C₃₀ fatty chains which may be indicated are the products Quatrisoft LM 200®, Quatrisoft LM-X 529-18-A®, Quatrisoft LM-X 529-18B® (C₁₂ alkyl) and Quatrisoft LM-X 529-8® (C₁₈ alkyl) sold by Amerchol and the products Crodacel QM®, Crodacel QL® (C₁₂ alkyl) and Crodacel QS® (C₁₈ alkyl) sold by Croda.

Amphoteric Associative Polymers

The amphoteric associative polymers may be chosen from those comprising at least one noncyclic cationic unit, for example, those prepared from or comprising 1 to 20 mol % of monomer comprising a fatty chain, for example 1.5 to 15 mol %, and further for example 1.5 to 6 mol %, with respect to the total number of moles of monomers.

In one embodiment of the present disclosure, the amphoteric associative polymers comprise or are prepared by copolymerizing:
1) at least one monomer of formula (XIX) or (XX): 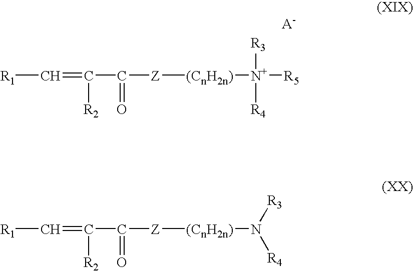
   wherein R₁ and R₂, which are identical or different, are chosen from a hydrogen atom and a methyl radical, R₃, R₄ and R₅, which are identical or different, are chosen from linear or branched alkyl radicals comprising 1 to 30 carbon atoms,

Z is an NH group or an oxygen atom,

n is an integer ranging from 2 to 5,

A⁻ is an anion resulting from an organic or inorganic acid, such as a methyl sulphate anion or a halide such as chloride or bromide;
2) at least one monomer of formula (XXI)
   R₆—CH═CR₇—COOH (XXI)
wherein R₆ and R₇, which are identical or different, are a hydrogen atom or a methyl radical; and
3) at least one monomer of formula (XXII):
   R₆—CH═CR₇—COXR₈ (XXII)
wherein R₆ and R₇, which are identical or different, are chosen from a hydrogen atom and a methyl radical, X is chosen from an oxygen atom and a nitrogen atom and R₈ is a linear or branched alkyl radical comprising 1 to 30 carbon atoms;
at least one of the monomers of formula (XIX), (XX) or (XXII) comprising at least one fatty chain.

In at least one embodiment, the monomers of formulae (XIX) and (XX) may be chosen from:
dimethylaminoethyl methacrylate, dimethylaminoethyl acrylate,
diethylaminoethyl methacrylate, diethylaminoethyl acrylate,
dimethylaminopropyl methacrylate, dimethylaminopropyl acrylate, and
dimethylaminopropylmethacrylamide, dimethylaminopropylacrylamide, these monomers optionally being quaternized, for example by a C₁-C₄ alkyl halide or a di(C₁-C₄ alkyl) sulphate.

For example, the monomer of formula (XIX) is chosen from acrylamidopropyltrimethylammonium chloride and methacrylamidopropyltrimethylammonium chloride.

The monomers of formula (XXI) of the present disclosure may, for example, be chosen from acrylic acid, methacrylic acid, crotonic acid and 2-methylcrotonic acid. In one embodiment, the monomer of formula (XXI) is acrylic acid.

The monomers of formula (XXII) of the present disclosure may be chosen from C₁₂-C₂₂, for example C₁₆-C₁₈, alkyl acrylates or methacrylates.

The monomers constituting the amphoteric polymers comprising a fatty chain of the disclosure may, in at least one embodiment, be already neutralized and/or quaternized.

The ratio of the number of cationic charges to the number of anionic charges may, for example, be 1.

As disclosed herein, the amphoteric associative polymers comprise from 1 to 10 mol % of the monomer comprising a fatty chain (monomer of formula (XIX), (XX) or (XXII)) and for instance from 1.5 to 6 mol %.

The weight-average molecular weights of the amphoteric associative polymers may range from 500 to 50 000 000 and, for instance, from 10 000 to 5 000 000.

The amphoteric associative polymers, as disclosed herein, may also comprise other monomers, such as nonionic monomers, for example C₁-C₄ alkyl acrylates or methacrylates.

In at least one embodiment of the present disclosure, amphoteric associative polymers are, for example, disclosed and prepared in International Patent Application WO 98/44012.

In yet another embodiment of the present disclosure, the amphoteric associative polymers are acrylic acid/(meth)acrylamidopropyltrimethylammonium chloride/stearyl methacrylate terpolymers.

Nonionic Associative Polymers

According to the present disclosure, the nonionic associative polymers useful herein may be, for example, chosen from:
(1) celluloses modified by groups comprising at least one fatty chain; non-limiting mention may be made, by way of example, of:
   hydroxyethylcelluloses modified by groups comprising at least one fatty chain, such as alkyl, arylalkyl or alkylaryl groups, or their mixtures, and wherein the alkyl groups may be, for instance, C₈-C₂₂ alkyl groups, such as the product Natrosol Plus Grade 330 CS® (C₁₆ alkyl) sold by Aqualon or the product Bermocoll EHM 100® sold by Berol Nobel,
   those modified by alkylphenol polyalkylene glycol ether groups, such as the product Amercell Polymer HM-1500® (nonylphenol polyethylene glycol (15) ether) sold by Amerchol.
(2) hydroxypropyl guars modified by groups comprising at least one fatty chain, such as the product Esaflor HM 22® (C₂₂ alkyl chain) sold by Lamberti or the products RE210-18® (C₁₄ alkyl chain) and RE205-1® (C₂₀ alkyl chain) sold by Rhône-Poulenc.
(3) copolymers of vinylpyrrolidone and of hydrophobic monomers comprising a fatty chain, of which may be mentioned, by way of example:
   the products Antaron V2160® or Ganex V216® (vinylpyrrolidone/hexadecene copolymer) sold by I.S.P.,
   the products Antaron V220® or Ganex V220® (vinylpyrrolidone/eicosene copolymer) sold by I.S.P.
(4) copolymers of C₁-C₆ alkyl methacrylates or acrylates and of amphiphilic monomers comprising at least one fatty chain, such as, for example, the methyl acrylate/oxyethylenated stearyl acrylate copolymer sold by Goldschmidt under the name Antil 208®.
(5) copolymers of hydrophilic methacrylates or acrylates and of hydrophobic monomers comprising at least one fatty chain, such as, for example, the polyethylene glycol methacrylate/lauryl methacrylate copolymer.
(6) polyether polyurethanes comprising, in their chain, both hydrophilic sequences of generally polyoxyethylene nature and hydrophobic sequences which can be aliphatic strings only and/or cycloaliphatic and/or aromatic strings.
(7) polymers comprising an aminoplast ether backbone having at least one fatty chain, such as the compounds Pure Thix® provided by Sud-Chemie.

In one embodiment of the present disclosure, the polyether polyurethanes comprise at least two lipophilic hydrocarbon chains comprising from 6 to 30 carbon atoms which are separated by a hydrophilic sequence, it being possible for the hydrocarbon chains to be pendant chains or chains at the end of the hydrophilic sequence. For instance, it is possible for at least one pendant chain to be provided. In addition, the polymer can comprise a hydrocarbon chain at one end or at both ends of a hydrophilic sequence.

The polyether polyurethanes may be polysequential, for instance in triblock form. The hydrophobic sequences can be at each end of the chain (for example: triblock copolymer comprising a central hydrophilic sequence) or distributed both at the ends and in the chain (polysequential copolymer, for example). These same polymers may also be graft polymers or star polymers.

The nonionic polyether polyurethanes comprising a fatty chain may be triblock copolymers, the hydrophilic sequence of which is a polyoxyethylene chain comprising 50 to 1000 oxyethylene groups. The nonionic polyether polyurethanes comprise a urethane bond between the hydrophilic sequences, hence the origin of the name.

By extension, the nonionic polyether polyurethanes comprising a fatty chain optionally include those, the hydrophilic sequences of which are bonded to the lipophilic sequences via other chemical bonds.

Non-limiting examples of nonionic polyether polyurethanes comprising a fatty chain which can be used in the invention include Rhéolate 205® comprising a urea functional group sold by Rheox or Rhéolates® 208, 204 or 212, and also Acrysol RM 184®.

Non-limiting mention may also be made of the product Elfacos T210® comprising a C₁₂-₁₄ alkyl chain and the product Elfacos T212® comprising a C₁₈ alkyl chain from Akzo.

The product DW 1206B® from Röhm & Haas comprising a C₂₀ alkyl chain and comprising a urethane bond, provided at a dry matter content of 20% in water, may also be used.

Non-limiting mention may be made of solutions or dispersions of these polymers, for instance in water or in an aqueous/alcoholic medium. Non-limiting mention may also be made, as examples of such polymers, of Rhéolate® 255, Rhéolate® 278 and Rhéolate® 244, sold by Rheox and of the products DW 1206F and DW 1206J provided by Röhm & Haas.

As disclosed herein, the polyether polyurethanes which may be used, for instance, are those described in the paper by G. Fonnum, J. Bakke and Fk. Hansen, Colloid Polym. Sci., 271, 380-389 (1993).

In yet another embodiment of the present disclosure, a polyether polyurethane may be used which is capable of being obtained by polycondensation of at least three compounds comprising (i) at least one polyethylene glycol comprising from 150 to 180 mol of ethylene oxide, (ii) stearyl alcohol or decyl alcohol and (iii) at least one diisocyanate.

Such polyether polyurethanes are sold for example by Röhm & Haas under the names Aculyn 46® and Aculyn 44® [Aculyn 46® is a polycondensate of polyethylene glycol comprising 150 or 180 mol of ethylene oxide, of stearyl alcohol and of methylenebis(4-cyclohexyl isocyanate) (SMDI), at 15% by weight in a matrix of maltodextrin (4%) and of water (81%); Aculyn 44® is a polycondensate of polyethylene glycol comprising 150 or 180 mol of ethylene oxide, of decyl alcohol and of methylenebis(4-cyclohexyl isocyanate) (SMDI), at 35% by weight in a mixture of propylene glycol (39%) and of water (26%)].

The at least one thickening polymer is present in the composition in an amount ranging from 0.1 to 5%, for example from 0.3 to 3%, by weight, with respect to the total weight of the composition.

The composition can additionally comprise at least one additive chosen from the cosmetic adjuvants and active principles commonly used in the hair field. These additives are chosen, for example, from vitamins, amino acids, oligopeptides, peptides, hydrolysed or nonhydrolysed and modified or unmodified proteins, enzymes, branched or unbranched fatty acids and alcohols, animal, vegetable or mineral waxes, ceramides and pseudoceramides, hydroxylated organic acids, UV screening agents, antioxidants and agents for combating free radicals, chelating agents, antidandruff agents, seborrhoea-regulating agents, soothing agents, ionic or nonionic surface-active agents, silicones, mineral, vegetable or animal oils, polyisobutenes and poly(α-olefin)s, additional fatty esters other than the esters of polyethylene glycol and the fatty acid mentioned above, hair coloring agents, such as direct dyes, oxidation dye precursors and pigments, acids, bases, plasticizers, fragrances, preservatives, inorganic fillers, pearlescent agents and glitter.

These additives are present in the composition according to the present disclosure in an amount ranging from 0 to 20% by weight for each, with respect to the total weight of the composition.

Of course, a person skilled in the art will take care to choose this or these additional cosmetic adjuvants and active principles so that the advantageous properties intrinsically attached to the arrangement and process in accordance with the disclosure are not, or not substantially, detrimentally affected by the envisaged addition or additions.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The following examples are intended to illustrate the present disclosure in a non-limiting manner.

### EXAMPLES

The following compositions (as % of active material) were prepared:

| INCI name | Ex. 1 | Ex. 2 |
|---|---|---|
| PVP | 5% | 6% |
| PEG-100 Stearate | | 2% |
| PEG-150 Distearate | 2% | |
| PEG-200 Glyceryl Stearate | 5% | 4% |
| Glycerol | | 2% |
| Propylene Glycol | 1.5% | |
| Sepigel 305 | 0.3% | 1% |
| (acrylamidopropanesulphonic acid | | |
| polymer as inverse emulsion) | | |
| Carbomer | | 1% |
| PEG-40 Hydrogenated Castor Oil | 0.5% | 0.5% |
| Ethanol | | 20% |
| Preservatives, Neutralizing agent, | q.s. | q.s. |
| Fragrance | | |
| Water | q.s. 100 | q.s. 100 |

These compositions exhibited good fixing properties and also persistence of these properties over time.

What is claimed is:

1. A cosmetic composition comprising, in a cosmetically acceptable medium:
at least one nonionic fixing polymer other than poly(vinylpyrrolidone/vinyl acetate) copolymers and
at least one ester of polyethylene glycol and of fatty acid of formula:
R₁CO—(OCH₂CH₂)ₙ₀—[OCH₂—CH(OR₂)—CH₂]ₙ₁—(OCH₂CH₂)ₙ₂—R₃
wherein
R₂ is chosen from hydrogen and a (CH₂CH₂O)ₙ₃COR₄ group;
n1 is an integer equal to 0 or 1;
n2 is an integer ranging from 2 to 300;
n3 is an integer ranging from 1 to 300;
n0 is an integer ranging from 0 to 300;
R₃ is chosen from hydrogen, an OH group and an R₅COO group;
R₁, R₄ and R₅ are chosen, independently of one another, from C₁₀ to C₃₀ alkyl groups and C₁₀ to C₃₀ alkylene groups.

2. A composition according to claim 1, wherein the at least one nonionic fixing polymer is chosen from polyalkyloxazolines, vinyl acetate homopolymers and copolymers, acrylic ester homopolymers and copolymers, acrylonitrile copolymers, styrene homopolymers and copolymers, polyamides, vinyllactam homopolymers and copolymers, nonionic polyurethanes and nonionic grafted silicone polymers.

3. A composition according to claim 2, wherein the vinyllactam homopolymers are polyvinylpyrrolidone homopolymers.

4. A composition according to claim 1, wherein the at least one nonionic fixing polymer is present in an amount ranging from 0.1 to 20%, by weight, relative to the total weight of the composition.

5. A composition according to claim 4, wherein the at least one nonionic fixing polymer is present in an amount ranging from 1 to 15%, by weight, relative to the total weight of the composition.

6. A composition according to claim 5, wherein the at least one nonionic fixing polymer is present in an amount ranging from 7 to 15% by weight relative to the total weight of the composition.

7. A composition according to claim 1, wherein the at least one ester of polyethylene glycol and of fatty acid is present in an amount ranging from 0.01% to 20% by weight with respect to the total weight of the composition.

8. A composition according to claim 7, wherein the at least one ester of polyethylene glycol and of fatty acid is present in an amount ranging from 0.1% to 15% by weight with respect to the total weight of the composition.

9. A composition according to claim 8, wherein the at least one ester of polyethylene glycol and of fatty acid is present in an amount ranging from 1% to 10% by weight with respect to the total weight of the composition.

10. A composition according to claim 1, wherein the at least one nonionic fixing polymer and the at least one ester of polyethylene glycol and of fatty acid are present in a ratio by weight of greater than 0.6.

11. A composition according to claim 10, wherein the ratio of the at least one nonionic fixing polymer to the at least one ester of polyethylene glycol and of fatty acid by weight ranges from 0.6 to 10.

12. A composition according to claim 1, wherein the composition further comprises at least one thickening agent.

13. A composition according to claim 12, wherein the at least one thickening agent is a thickening polymer.

14. A composition according to claim 13, wherein the at least one thickening polymer is an associative anionic polymer comprising at least one hydrophilic unit of unsaturated olefinic carboxylic acid and at least one hydrophobic unit of (C₁₀-C₃₀)alkyl ester of unsaturated carboxylic acid.

15. A cosmetic composition according to claim 13, wherein the at least one thickening polymer is present in an amount ranging from 0.01 to 10% by weight, with respect to the total weight of the composition.

16. A cosmetic composition according to claim 15, wherein the at least one thickening polymer is present in an amount ranging from 0.1 to 5% by weight, with respect to the total weight of the composition.

17. A cosmetic composition according to claim 16, wherein the at least one thickening polymer is present in an amount ranging from 0.3 to 3% by weight, with respect to the total weight of the composition.

18. A composition according to claim 1, further comprising at least one additive chosen from vitamins, amino acids, oligopeptides, peptides, hydrolysed or nonhydrolyzed and modified or unmodified proteins, enzymes, branched or unbranched fatty acids and alcohols, animal, vegetable or mineral waxes, ceramides and pseudoceramides, hydroxylated organic acids, UV screening agents, antioxidants and agents for combating free radicals, chelating agents, antidandruff agents, seborrhoea-regulating agents, soothing agents, ionic or nonionic surface-active agents, silicones, mineral, vegetable or animal oils, polyisobutenes and poly(α-olefin)s, additional fatty esters, hair coloring agents, acids, bases, plasticizers, fragrances, preservatives, inorganic fillers, pearlescent agents and glitter.

19. A composition according to claim 18, wherein the hair coloring agents are chosen from direct dyes, oxidation dye precursors and pigments.

20. A composition according to claim 1, wherein said composition is in the form of a spray.

21. A composition according to claim 1, wherein said composition is in the form of a gel.

22. A composition according to claim 1, wherein said composition is in the form of a foam.

23. A method for fixing, shaping or providing form retention to a hairstyle, comprising applying to hair a cosmetic composition comprising, in a cosmetically acceptable medium, at least one nonionic fixing polymer other than poly(vinylpyrrolidone/vinyl acetate) copolymers and at least one ester of polyethylene glycol and of fatty acid of formula:
R₁CO—(OCH₂CH₂)ₙ₀—[OCH₂—CH(OR₂)—CH₂]ₙ₁—(OCH₂CH₂)ₙ₂—R₃
wherein
R₂ is chosen from hydrogen and a (CH₂CH₂O)ₙ₃COR₄ group;
n1 is an integer equal to 0 or 1;
n2 is an integer ranging from 2 to 300;
n3 is an integer ranging from 1 to 300;
n0 is an integer ranging from 0 to 300;
R₃ is chosen from hydrogen, an OH group and an R₅COO group;
R₁, R₄ and R₅ are chosen, independently of one another, from C₁₀ to C₃₀ alkyl groups and C₁₀ to C₃₀ alkylene groups.

24. A method for conferring good hold over time on a hair style comprising applying to the hair a cosmetic composition comprising, in a cosmetically acceptable medium, at least one nonionic fixing polymer other than poly(vinylpyrrolidone/vinyl acetate) copolymers and at least one ester of polyethylene glycol and of fatty acid of formula:
R₁CO—(OCH₂CH₂)ₙ₀—[OCH₂—CH(OR₂)—CH₂]ₙ₁—(OCH₂CH₂)ₙ₂—R₃
wherein
R₂ is chosen from hydrogen and a (CH₂CH₂O)ₙ₃COR₄ group;
n1 is an integer equal to 0 or 1;
n2 is an integer ranging from 2 to 300;
n3 is an integer ranging from 1 to 300;
n0 is an integer ranging from 0 to 300;
R₃ is chosen from hydrogen, an OH group and an R₅COO group;
R₁, R4 and R₅ are chosen from, independently of one another, C₁₀ to C₃₀ alkyl groups and C₁₀ to C₃₀ alkylene groups.
